(19) United States
(12) Patent Application Publication
(10) Pub. No.: US 2006/0223167 A1
(43) Pub. Date: Oct. 5, 2006

(54) Biochip support comprising thin layers of sol-gel material and production method thereof

(76) Inventors: Patrick Chaton, Theys (FR); Stéphane Getin, Grenoble (FR); Francoise Vinet, Grenoble (FR); Philippe Pegon, Saint-Etienne (FR); Chantal Germain, Montrond Les Bains (FR)

Correspondence Address: Eugene Lieberstein;Anderson Kill & Olick, 1251 Avenue of the Americas, New York, NY, 10020, US

(21) Appl. No.: 10/555,580

(22) PCT Filed: May 6, 2004
(86) PCT No.: PCT/FR04/01088

§ 371 Date: Nov. 4, 2005

(30) Foreign Application Priority Data
May 6, 2003 (FR) 03/50146

(51) Int. Cl. C12M 1/34; H01L 21/00

(52) U.S. Cl. 435/287.2; 438/1; 977/702; 977/924

(57) ABSTRACT

The invention relates to a biochip support comprising a substrate supporting at least one porous layer of material on a first face, the said layer being designed to fix biological molecules onto the said layer and in the volume of this layer, the said support being characterized in that the said layer is a thin optical layer of material prepared by the sol-gel method and for which the refraction index is less than the refraction index of the substrate. The invention also relates to a process grafting of biological molecules onto and into the thin layer of material prepared by the sol-gel method on the first face of the biochip support. This process comprises the following steps: a sol is prepared that will provide the sol-gel material, biomolecules are incorporated into the material during its preparation, biomolecules are grafted into the material during its preparation, a thin layer of the said sol is deposited on the first face of the substrate, the thin layer of sol-gel material is obtained starting from the thin layer of sol. Finally, one particular embodiment of the grafting process also comprises a structuring step of the thin layer of sol-gel material to obtain a network of pads or wells over all or part of the biochip support.

### TECHNICAL DOMAIN

The invention relates to biological Microsystems. More particularly, it relates to the production of a biochip system for which detection is based on the collection of a fluorescence signai.

This biochip or biosensor support may be used for the production of biochip supports compatible with grafting of nucleic acids (DNA biochips, RNA biochips), amino acids (protein chips, immunological chips), and cellular biochips used particularly in transfection studies or chemotaxis studies.

The field of the invention also covers all types of molecular recognition reactions (antibodies/antigen, protein-sugars, etc.).

The invention also relates to a process for grafting biological molecules in the support according to the invention.

### STATE OF PRIOR ART

The DNA biochips or biosensors technology is widely used in biomedical analyses, for example such as the expression of genes, detection of mutations or polymorphism, sequencing and discovery of genes (see document [1] referenced at the end of this description).

These biochips are composed of microscopic networks of biological molecules immobilised on solid supports. Several approaches can be used for preparation of these micronetworks.

Firstly, biological molecules can be directly synthesised on a substrate (see documents [2], [3], [4], [5]). However, by proceeding in this manner, the lengths of the oligonucleotides obtained are within the range of 10 to 60 mers. The main problem with this technology is the limitation in the maximum length of the probes thus synthesised.

Biological molecules can also be pre-synthesised and then deposited on delimited areas of the support by means of microrobots that will be either passive (<<pin>> type) or active (piezoelectric or inkjet type) (see document [6]). A large range of molecules can thus be deposited by this method, for example such as oligonucleotides, products derived from DNA PCR amplification and proteins.

Similarly, different approaches are possible to couple biological molecules with the support.

Electrostatic coupling on polyLysine (document [7]) or hydrophobic interactions (document [8]) can be used. However, this type of bond does not enable a precise analysis under some temperature or stringency conditions of hybridising solutions.

Biological molecules can also be coupled to the support using covalent bonds. This approach makes it possible to better optimise hybridising conditions and therefore the sensitivity of micronetworks, since the molecules are grafted onto the support covalently and irreversibly (documents [9], [10], [11], [12]).

Coupling can also be done with oligonucleotides modified in 5′ by a pyrrol group that is deposited on electrodes by electrocopolymerisation with pyrrol (document [13]).

However, grafting is done in two dimensions on a plane support, regardless of which of the methods described above is used.

A. Mirzabekov proposed to use polyacrylamide gels and to graft molecules in three dimensions in the volume of this material, in order to increase the local density of biological molecules on the biochips (documents [14], [15], [16]). This principle is also used for localised in situ enzymatic reactions, antigen/antibody recognitions (document [17]), PCR amplifications of immobilised oligonucleotides (document [18]) or detection of the production of bacterial toxins.

By using polyacrylamide gels like those described in the patent deposited by A. Mirzabekov (document [19]), a greater density of grafted biomolecules can be obtained than is possible in prior art. But in this process, only the organic chemistry reactions are used to couple the biomolecules. In particular, no silane type coupling agent is used to graft biomolecules onto the gel.

Furthermore, no attempt is made to improve the fluorescence signal emanating from the biochip or more particularly, the [fluorescence signal/noise] ratio. In particular, the polyacrylamide monolayer does not perform any optical function.

Furthermore, this monolayer is thick: its thickness is about 30 micrometers.

Concerning the sol-gel materials themselves, a number of these materials have already been used mainly for encapsulation of proteins and for studying their functions. Much work is now being published on the influence of this encapsulation on the structure, function, accessibility, conformation and thermodynamic stability of proteins (documents [20], [21], [22]). In particular, it is known that appropriate chemical modification of sol-gels enables a greater covalent grafting ratio of proteins and/or an improvement in stability properties. Comparative stability studies of immobilised antibodies have shown that better results on the sol-gel are possible than with covalent grafting on glass (see document [23]). For example, two or three times higher grafted protein ratios can be obtained with sol-gels modified by chloropropyl or aminopropyl functions than is possible on glass (document [24]).

Some observations can be made on these previously published studies.

Firstly, it can be seen that the number of tested materials is very limited. Tested materials are usually limited to thick silica matrices (>1 mm) prepared based on tetraethoxysilane (TEOS) or tetramethoxysilane (TMOS). Variants are mostly related to modification of the matrix by:
introduction of polymer (PVA, PEG with low molecular weight) (document [21]) or diffusion of ions to vary the local polarity in the pores,
introduction of ligands to fix the biomolecules,
diffusion of solvent (for example glycerol (document [22])) to modulate the local viscosity.

Therefore, the state of the art has not fully exploited the versatility of microstructural and physicochemical properties of sol-gel materials, and the different means of introduction and grafting of biomolecules onto or in a sol-gel layer.

Furthermore, the potential advantage of thin layers (in other words layers with a thickness of the order of 0.1 μm) has been mentioned several times but apparently has never previously been proven, except by Zink et al. (document [25]). Nevertheless, this advantage is in no way motivated by the optical function of the layer. In particular, the optical properties of thick (>1 μm) and thin sol-gel layers used for encapsulation of proteins, regardless of whether or not they are chemically modified, have not been studied or quantified. The authors were satisfied simply if the gel was sufficiently transparent to enable comparative measurements of fluorescence signals. The concept that the optical characteristics of these layers might contribute towards optimising the fluorescence detection signal is not mentioned.

### PRESENTATION OF THE INVENTION

The purpose of the invention is to provide a biochip support that can have a higher local density of grafted biological molecules than is possible with prior art, but which is also capable of obtaining an improved fluorescence detection signal (more precisely, an improved signal to fluorescence detection noise ratio).

This and other purposes are achieved according to the invention by a biochip support comprising a substrate supporting at least one porous layer of material on a first face, the said layer being designed to fix biological molecules onto the said layer and in the volume of this layer, the said support being characterised in that the said layer is a thin optical layer of material prepared by the sol-gel method and for which the refraction index is less than the refraction index of the substrate.

Material layers obtained by the sol-gel method have a large developed grafting surface area due to their very high porosity compared with a layer of the same nature obtained by deposition using other conventional techniques (PVD, IBS, CVD). This high porosity makes it possible to graft large concentrations of biological molecules in three dimensions, and at the same time to significantly increase the fluorescence signal. Furthermore, considering the efficiency of in-depth grafting of layers of sol-gel material, the thickness of the sol-gel material to be deposited on the substrate is less than the thickness of gels conventionally used in prior art. The thickness of layers used in the invention can thus vary from 0.1 to 5 μm, depending on the nature of the deposited layer and the density of the probes to be grafted to it. These layers may be deposited in one or several steps using techniques described later on. Therefore, the invention is within the domain of thin layers; unlike prior art which applied to thick layers (more than 20 μm and nominally 30 μm).

Therefore, we will now use the optical characteristics of thin sol-gel layers deposited on the substrate. These optical characteristics of thin layers will also be optimised so as to increase the detected fluorescence signal after grafting the marked molecule on or in the sol-gel layer in question.

Advantageously, due to the choice of precursors and the method of synthesising the thin layer of sol-gel material, a very low intrinsic fluorescence of the layer at the wavelength of interest can be obtained, in other words at excitation wavelengths that will be sent to the device later to study the fluorescence of fluorophores fixed in or to the surface of the thin layer.

Furthermore, the layer of sol-gel material may have low absorption in the UV and the visible ranges, which can limit the contribution of non-radiative losses. The result is an improvement in the energy balance in favour of the emission of fluorescence in free space. With this characteristic, the quantity of light emitted is greater than in prior art. Therefore signals collected by biochip read scanners based on the invention will have an improved signal to noise ratio.

Furthermore, the sol-gel layer can advantageously have a low surface roughness, which will limit the diffusion of fluorescence light.

For these first three points, the thin sol-gel layers used by this invention will improve performances compared with prior art. Therefore, the fluorescence signal is increased by arranging a thin layer of sol-gel material on the substrate.

According to one particular embodiment, the support according to the invention also comprises at least one thin optical layer of material prepared by a sol-gel method supported by a second face of the substrate opposite the first face, the said thin layer supported by the second face having a refraction index lower than the refraction index of the substrate. In other words, the device according to the invention comprises one or a plurality of thin layers on one face of the substrate and one or a plurality of thin layers on the opposite face.

According to another embodiment, the support according to the invention comprises a stack of dielectric thin layers forming a Bragg mirror inserted between the substrate and the thin layer of material prepared by the sol-gel method. This particular configuration will make it possible to increase the excitation field inside the thin layer of sol-gel material. This is particularly useful when it is required to study fluorescence of fluorophores grafted in or on the surface of the sol-gel layer, because the effect of this configuration is to increase excitation of the said fluorophores and therefore to increase the quantity of light emitted in the superstrate. Note that a Bragg mirror is a successive stack of several thin dielectric layers with different refraction indexes n1 or n2. The thickness of each of these layers is equal to λ/(4n), where n can be the value n1 or n2. The variable λ corresponds to the wavelength where we want to have a maximum reflection for the Bragg mirror.

Material layers prepared by a sol-gel method are compatible with most substrates (mineral or organic) regardless of their chemical nature and their optical, mechanical or thermal properties. Advantageously, the substrate will be formed using a material chosen from among the group including glasses, polymers and semiconductors. Therefore, it could be envisaged to make biochip supports on a glass, silicon, or plastic substrate. Obviously, any other material could be chosen provided that the layers of sol-gel material are compatible with the chosen substrate.

According to one particular embodiment, the material prepared using the sol-gel method has a purely inorganic composition. According to a second embodiment, it is composed of an inorganic fraction and an organic fraction.

If the material is composed of both an inorganic fraction and an organic fraction, the inorganic fraction will advantageously be larger than the organic fraction. Advantageously, the said fraction will confer its cohesion to the sol-gel material, regardless of whether or not the inorganic fraction is in the majority.

This material will advantageously comprise at least one compound chosen from among the following, regardless of whether the composition of the material is purely inorganic, or is both inorganic and organic:
an oxide MxOy, where M is chosen from among the group composed of Si, Al, Zr, Ti and Ta,
an -M-O-M′- type compound, where M and M′ are chosen from among the group composed of Si, Al, Zr, Ti and Ta.

For example, it would thus be possiable to have materials such as SiO2, TiO2, Ta2O5, ZrO2 and/or mixes of two or more of these oxides.

Advantageously, when the material prepared using a sol-gel method comprises an -M-O-M′- type compound, M is Si and M′ is Zr or Ti.

For example, in this type of layer, it would be possible to use free M-OH sites as reactive sites for subsequent grafting of oligonucleotide probes, after silanisation of these sites or probes.

When the sol-gel material is composed of an inorganic fraction and an organic fraction, the said material has particular characteristics.

According to one particular embodiment, the organic fraction is a polymer, the said polymer remaining free or being weakly bonded to the elements forming the inorganic fraction. In particular, the organic fraction is the result of the addition of an organic polymer into the inorganic colloidal suspension that will form the sol-gel material. By proceeding in this way, and after the steps to deposit and gel the material on the substrate, the polymer is trapped in the meshes of the inorganic network. This polymer may have a plurality of functions, and in particular partial filling of the natural porosity of the layer, thus making it possible to modulate the natural porosity of the layer and/or the size of the pores, and incorporation of reactive organic groups into the layer to graft oligonucleotide probes.

According to another embodiment, the organic fraction is the result of incorporating a silane X—R2-Si(OR1)n into the inorganic fraction. In particular, this incorporation is done during synthesis of the sol and the silane is bonded to salts or metallic alcoxides of the inorganic fraction (remember that the sol denotes a colloidal suspension of particles in a liquid). The result is then creation of an M-O—Si(R2X)—O-M- type composite network.

Advantageously, R1 will be chosen from among the group comprising —CH3, —C2H5, nPr, iPr or tBu, R2 will be an aliphatic chain with length p-CH2, preferably without an ether function —CH2-O—CH2-, where p is between 2 and 10, and X will be a reactive terminal organic group chosen from among the group comprising —OH, —COOH, —CH═O, —NH2, —Cl, -epoxy, -glycidoxy, —CH═CH2, -acryl or -methalcryl.

Note that the length of the aliphatic chain may be used to modulate the size of cavities in the network, in order to collect an oligonucleotide probe with a determined length. The group X may be used to graft an oligonucleotide that is itself functionalised.

Conventional hydrolysis and condensation steps may be used for precursors in solution, followed by a sol-gel transition, to synthesise layers of the sol-gel material according to the invention.

The sol-gel layer is deposited starting from a liquid product that is a colloidal suspension of the compounds mentioned above, in other words it is composed of species with a size of between 5 nm and 100 nm.

This colloidal suspension is prepared by hydrolysis followed by controlled condensation of salts or alcoxides of metals or metalloids M. Finally, the carrier medium is an organic solvent in order to facilitate the film deposition and drying step.

Elements present in the colloidal suspension may be particles in quasi-spherical or platelet form, or as oligomers, or they may be a mix of particles onto which oligomers will be grafted. In all cases, the characteristic dimension of the particles and/or the oligomers will be relatively monodispersed and within the range of sizes described above.

In fact, the nature, size and shape of these species in suspension will confer the microstructure of the layer of sol-gel material after deposition and gelling. These species will determine the total porosity and the pore size in the sol-gel layer. In general, the structure of the layer will be of the oligomeric type.

In producing the sol-gel material, the pore size in the thin layer of material prepared by the sol-gel method can be controlled. Advantageously, pore size in the thin layer of material prepared by the sol-gel method will be between 5 nm and 100 nm, and the total porosity will be between 1% and 50%.

There are different methods of optimising the pore size and the total porosity of the sol-gel layer.

Firstly, the following parameters can be varied for sol-gel layers with a particulate structure:
the particle size determined by material synthesis conditions (concentration, hydrolysis ratio, sol curing time),
the form of the particles determined as a function of the precursor (nature of the metal M, nature of the salt or alcoxide) and material synthesis conditions,
the compactness of the stack of particles in the layer (compact stacking such as hard spheres or a string of particles forming a network introducing cavities of a certain dimension in which probes to be grafted can be fitted),
the use of a filling agent intimately mixed with or covalently bonded to the particles.

The following parameters may be varied for sol-gel layers with an oligomeric structure:
the size of the oligomers,
the compactness of the stack of oligomers and/or the natural size of the network after gelling,
possibly densification of the layer after being deposited, by a thermal or other method,
incorporation of variable length lateral organic grafts used as spacers into the network.

By varying these parameters, the size of the pores and the total porosity of the thin layer obtained by the sol-gel method can be controlled within the ranges mentioned above. This also makes it possible to control the density of grafted probes within a very wide range of concentrations. Therefore, industrial manufacturing of standard chips and <<tailored>> chips becomes possible for many specific applications, so that the fluorescence signals can be normalised between probes with different natures and/or lengths.

Furthermore, the refraction index of the said layer can be modulated as a function of the sol-gel matrix chosen to form the layer of sol-gel material. The layer of sol-gel material can thus have a refraction index within the range 1.2 to 2.1. This makes it possible to optimise the fluorescence signal detected depending on the detection technique used (microscopy in near field or in far field, ellipsometry, etc.) and depending on the grafting method (surface or in-depth). In the case of this invention, conditions for production of the layer are obtained such that its refraction index (n) can be made lower than the refraction index of the exposed substrate. It is advantageous if the following relation is satisfied for the purposes of this invention:
n(sol-gel layer)<n(substrate)

In the special case in which a thin sol-gel layer is used on an optical multilayer (Bragg mirror), this relation will be generalised in the following form:
Y(sol-gel layer)<Y(multilayer)

where Y is the optical admittance of the wave in the thin layer of sol-gel material or the Bragg mirror.

The purpose of the support according to the invention is to make biochips or any other biological analysis system. To achieve this, the biological molecules or biomolecules are grafted onto and into the thin layer(s) obtained by the sol-gel method according to the invention. To achieve this, the biological molecules or biomolecules are grafted onto and into the thin layer of material prepared by the sol-gel method on the first face of the support for a biochip according to the invention by performing the following steps:
a sol is prepared that will provide the sol-gel material,
biomolecules are incorporated into the material during its preparation,
biomolecules are grafted into the material during its preparation.
a thin layer of the said sol is deposited on the first face of the substrate,
the thin layer of sol-gel material is obtained starting from the thin layer of sol.

According to one special case, the biomolecules incorporated into the material during its preparation are silanised biomolecules so that they can be grafted.

In this case, biomolecules can be incorporated in different ways.

According to a first embodiment, biomolecules are incorporated into the said thin layer by diffusion when it is in the form of a dry gel. The following procedure could be used to graft the biological molecules onto and into the thin layer of material prepared by the sol-gel method on the first face of the support for the biochip:
synthesise the sol-gel material that will constitute the thin layer,
silanise or functionalise the biomolecules to be grafted,
deposit the sol-gel material on the substrate in the form of a thin layer,
incorporate the biomolecules in the dry gel by diffusion,
and graft the biomolecules on the sol-gel matrix.

According to a second embodiment, biomolecules are incorporated into the said thin layer when it is in the form of a wet gel, the biomolecules being grafted while the gel is drying (remember that the gel denotes a solid three-dimensional network trapping the liquid (in its meshes)).

According to a third embodiment, biomolecules are incorporated to the sol-gel material in the liquid state, the biomolecules being grafted in the sol. The grafted sol will then only have to gel and dry at the time of the deposition. In other words, biomolecules are incorporated in the sol-gel material when the sol-gel material is in the sol form, the biomolecules being grafted in the sol before the thin layer is deposited in the liquid state.

According to another special case, the sol preparation step includes a functionalisation step that will result in a functionalised sol-gel material enabling grafting of biomolecules after they have been incorporated in the thin layer.

In this case, biomolecules can be incorporated in different ways.

According to a first embodiment, the biomolecules are incorporated into the thin layer when the thin layer is in dry gel form. Bioloaical molecules are grafted onto and into the thin layer of material prepared by the sol-gel method on the first face of the support for a biochip using the following procedure:
synthesise the sol-gel material including its functionalisation so that biomolecules can be grafted,
deposit the sol-gel material on the substrate in the form of a thin layer,
incorporate the biomolecules in the dry gel,
and graft the biomolecules.

According to a second embodiment, the biomolecules are incorporated into the thin layer when it is in wet gel form. In this case, the biomolecules can be grafted while the gel is drying.

According to a third embodiment, the biomolecules are incorporated into the sol-gel material when it is in sol form, the biomolecules then being grafted into the sol before the thin layer is deposited. Therefore in this case, the biomolecules will be grafted before the sol gels and the layer will then be deposited and dried later.

According to a fourth embodiment, the biomolecules are also functionalised and are then incorporated and grafted in the sol before it is deposited in a thin layer on the support. The following procedure could be used for this particular grafting:
synthesise the sol-gel material including its functionalisation so that biomolecules can be grafted,
functionalise the biomolecules,
incorporate the biomolecules in the liquid sol,
graft the biomolecules,
deposit the sol containing the grafted biomolecules on the support,
wait until the sol has gelled and then dry the gel.

According to one particular embodiment of the invention, the layer of sol-gel material can be structured and given a particular configuration. This can be done using the grafting method as described above, and by adding a structuring step of the thin layer of sol-gel material to it to obtain a network of pads or wells over all or some of the biochip support. Advantageously, these pads or wells will be circular or square in shape.

Advantageously, the pads or wells will have a characteristic size of between 10 to 200 micrometers, and will be at a spacing of 50 to 200 micrometers.

Advantageously, the network of pads or wells is made using at least one of the techniques chosen from among etching, peeling, micro-machining of the layer of material prepared by a sol-gel method or by direct deposition of a structured layer of material prepared by the sol-gel method by local micro-distributions. The choice of the structuring technique will preferably be made as a function of the characteristics of the sol-gel layer. In particular, the following will be considered:
coupling of the sol-gel layer to the substrate and its mechanical strength,
the proportion of the inorganic fraction and the organic fraction,
the chemical resistance of the inorganic fraction, mainly to mineral acids and bases, and of the organic fraction, particularly to organic solvents.

If the layer comprises a large fraction of photocrosslinkable organic, it will be possible to use direct forming or development by etching after photocrosslinking through a mask. If etching is used, it will be possible to use a lift-off type protocol, for example. The peeling technique will be used in preference if the layer of sol-gel material is weakly bonded to the substrate. Micro-machining of the layer of material prepared by the sol-gel method will be used in preference if the layer has a weak to moderate mechanical strength. It will be possible to use the ink-jet technique or the pin and ring technique, for example, if the structured layer is deposited directly by local microdistributions.

In summary, this invention can be used to deposit thin layers based on SiO2, TiO2, Ta2O5, ZrO2 by a sol-gel method, onto substrates of different natures (glass, polymer or semiconductor). Considering the particular optical properties of these thin sol-gel layers (low refraction indexes and extinction coefficient) and their physicochemical properties (porosities, interface state), fluorescence signals different from signals possible with the state of the art are obtained. The low thickness of the sol-gel layer deposited on the substrate, combined with a low refraction index and a low absorption coefficient, are responsible for the particular optical functions of the layer, in addition to increasing its graftability.

### BRIEF DESCRIPTION OF THE DRAWINGS

The invention will be better understood and other advantages and special features will become clear after reading the following description given as a non-limitative example accompanied by the attached drawings among which:
FIG. 1 is a graph showing the variation of the fluorescence signal originating from a biochip composed of a support according to the invention as a function of the thickness of the thin layer of sol-gel material arranged on the said support,
FIG. 2 is a diagram of a support according to a special case of the invention, the said support being composed of a substrate, in which the two opposite faces support a thin layer of sol-gel material,
FIG. 3 is a diagram showing a substrate supporting a multi-layer on one face, on which a thin layer of sol-gel material is deposited.

### DETAILED PRESENTATION OF A PARTICULAR EMBODIMENT

Firstly, the behaviour of a sol-gel monolayer deposited on a microscope slide under fluorescence will be optimised.

This experiment will be carried out considering a glass substrate with an index of 1.52 comprising a thin layer on which a fluorophore is grafted. The fluorophore considered is CY3 (excitation=543 nm, emission=580 nm). Note that the dipole moment of this type of molecules tends to orient itself parallel to the surface (see document [26]).

The experiment was carried out for two values of refraction indexes of the thin layer (1.2 and 1.4), the two indexes being chosen less than the index of the substrate, and for a digital aperture of the microscope equal to 0.5.

A laser excitation beam with an intensity lex is applied to the thin layer at the excitation wavelength of the fluorophore and fluorescence is observed.

Under these conditions, the fluorescence behaviour of the device according to the invention as a function of the thickness of the thin layer follows the variation shown in FIG. 1. Curves 1 represents the total intensity and curve 2 represents the intensity obtained for a digital aperture of 0.5, with an index n=1.2; curve 3 represents the total intensity and curve 4 represents the intensity obtained for a digital aperture of 0.5, with a index of n=1.4. As expected, FIG. 1 indicates that by choosing a material with a lower index (n=1.2), emission of fluorescence in the superstrate (incident medium) is given preference. Thus, when n=1.2, the efficiency with a sol-gel monolayer about a hundred nanometers thick is 3.5 times better than the efficiency obtained with a microscope slide with index n=1.52.

This thin layer of sol-gel material about a hundred nanometers thick with a low index can reinforce the transmitted signal, when it is deposited on the two faces of a substrate. The waves coupled by the fluorophores inside the acceptance cone propagate according to Descartes laws. But the waves are affected by an attenuated Fresnel reflection due to the presence of a low refraction index at the surface. FIG. 2 shows that when a light beam 6 is directed onto a face of the substrate 5 with fluorophores on its surface, the result is emission 7 in the superstrate and emission 8 in the substrate. When the fluorophores are grafted in a thin layer 9 of sol-gel material with an index less than the index of the substrate and deposited on one of the faces of the substrate, the result is an emission 17 in the superstrate greater than the emission 7 and an emission 18 in the substrate greater than the emission 8. Advantageously, as shown in this Figure, a second thin layer 9 of sol-gel material is deposited on the face opposite to the face of the substrate 5 on which a first layer 9 is already present. The presence of this thin layer 9 with a lower index than the substrate will cause the appearance of a phenomenon reducing reflection of the light beam 6.

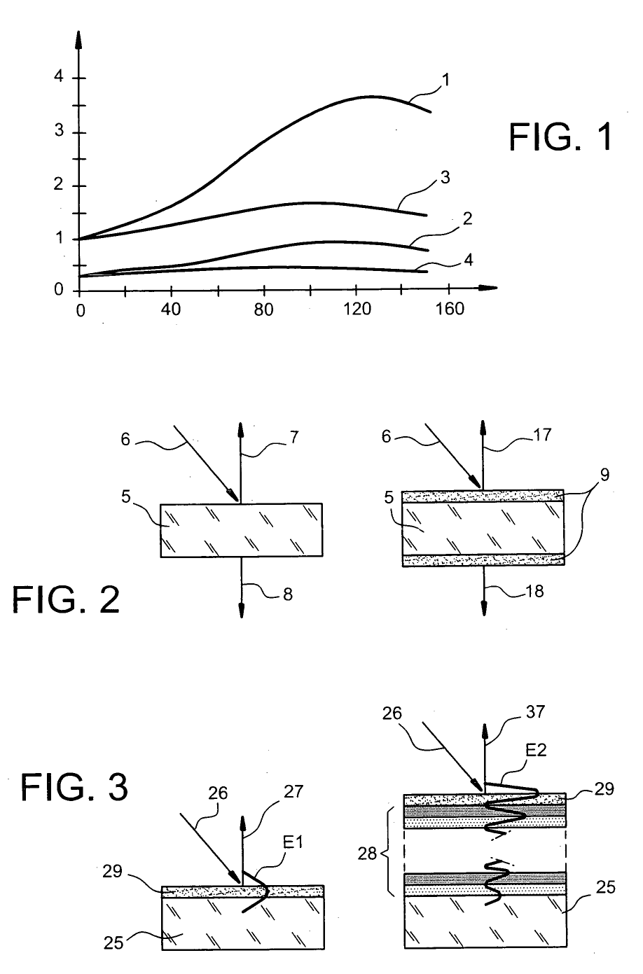

According to another embodiment, the thin layer 29 with a low refraction index can also be deposited on an optical multilayer 28 of the Bragg mirror type (see in FIG. 3). It will then be noted that the excitation field inside the thin sol-gel layer is increased when an incident beam 26 is directed onto the support. We get E1<E2, where E1 and E2 are the excitation field of the substrate and of the thin layer respectively. This particular configuration can therefore increase fluorescence emitted in the superstrate because the emission 37 in the superstrate is greater than emission 27 in the superstrate without multilayer.

Models are made comparing four types of stacks, to illustrate the advantage of the device according to the invention and its different variants:
case 1 is a substrate alone,
case 2 is a substrate and a stack of type 5 (HB) thin layers, where H is TiO2 and B is SiO2,
case 3 is a substrate and a stack of type 4 (HB)(HB′) thin layers, where B′ is a sol-gel material with refraction index n=1.3,
case 4 is a substrate and a stack of type 4(HB)(HB′) thin layers, where B′ is a sol-gel material with refraction index=1.22.

In carrying out this study, CY3 fluorophores will be used located at an altitude of 2 nm and at an orientation of 90° from the surface of the support.

The excitation and emission wavelengths of the laser sent to the samples are 543 nm and 580 nm respectively. Note that the refraction indexes of SiO2 and TiO2 are 1.46 and 2.2 respectively, the index of the superstrate (in other words the index of the light arrival medium) is equal to 1 and the index of the substrate is 1.52. The digital aperture (DA) of the microscope used for observation of fluorescence of the microscope is 0.5.

Simulations on the CEA-LETI <<fluoplus>> software give the following results:

| | | Intensity in a | Intensity in the |
|---|---|---|---|
| | Excitation field | 0.5 DA | superstrate |
| | (arbitrary unit) | (arbitrary unit) | (arbitrary unit) |
| Case 1 | 0.63 | 0.03 | 0.10 |
| Case 2 | 3.02 | 0.73 | 2.04 |
| Case 3 | 3.25 | 0.86 | 2.44 |
| Case 4 | 3.33 | 0.92 | 2.64 |

These results can be compared with case 1 considered as a reference, to give:

| | Excitation | | Intensity in |
|---|---|---|---|
| | field | Intensity in a | the superstrate |
| | (arbitrary | 0.5 DA | (arbitrary |
| | unit) | (arbitrary unit) | unit) |
| Case 1 | 1 | 1 | 1 |
| Case 2 | 5 | 27 | 20 |
| Case 3 | 5 | 32 | 24 |
| Case 4 | 5 | 34 | 26 |

These results show that with a sol-gel with an index lower than silica, the total emission of fluorescence can be increased without modifying the bleaching (excitation intensity), and this phenomenon is further accentuated if there is an intermediate Bragg mirror type optical treatment between the substrate and the layer with the low surface index. This is an important element that is not present in prior art.

We will now describe details of an embodiment based on covalent grafting of oligonucleotide probes onto a thin layer of silica made using the sol-gel method, to illustrate this invention.

Firstly, we will make the sol-gel material by applying conventional steps of hydrolysis and condensation of precursors in solution, followed by a sol-gel transition as described above.

When the sol-gel material has been made starting from a liquid treatment solution, it can be applied uniformly in a thin layer over the entire substrate using one of the following techniques:
dipping or dipping-shrinkage,
centrifuging,
<<meniscus coating>> type of horizontal coating, in other words deposition by pulling,
atomisation.

The low viscosity and uniformity of treatment solutions also enable <<spotting>> micro-distribution techniques, in other words deposition of liquid drops with a volume of between a few tens and a few hundreds of picolitres. Another applicable technique is adaptation of an ink jet coating. Therefore, pads of sol-gel material could also be made on the substrate directly by local distribution. This procedure is particularly attractive if the grafting method used is based on the micro-reactor principle. Furthermore, it eliminates the structuring step of a layer deposited over the entire biochip and provides means of local deposition on well wafers.

Once the thin layer has been deposited on the substrate, it may be necessary to structure the said thin layer, the end purpose being to create pads or wells on the biochip with a circular or square geometry, with dimensions of between 10 and 200 μm and at a spacing of 50 to 200 μm.

In the case of local micro-distribution using <<spotting>> or <<ink jet coating>> techniques, the structuring of the thin layer is a direct result of the deposition.

Deposition conditions such as preparation of the substrate, the size and spacing of micro-droplets, drying and gelling are studied such that the droplets do not coalesce before gelling, and that the dimensions are as required after gelling.

In the case in which the sol-gel layer is deposited firstly over the entire substrate using one of the techniques mentioned above, structuring will be obtained by one of the following techniques depending on the physicochemical nature of the layer and its mechanical properties:
etching, for example using a lift-off type protocol using an insolation mask, possibly a sacrificial layer, a mineral acid or base, and an aqueous or organic solvent,
in the case of a layer with a low density and/or only weakly bonded to the substrate, simple mechanical peeling of the layer is made using an adhesive mask that can obtain a network of micro-pads over the entire chip in a single operation,
automated micro-machining in the case of a layer with a weak to moderate mechanical strength,
in the case of a layer comprising a high organic photocrosslinkable fraction, direct shaping or development by etching is possible after photocrosslinking through a mask.

We will now consider the probe preparation step. The fluorophore F and the graft G will be grafted onto the oligonucleotide probe N, and the graft G may be a silane S or an organic sequence R1.

When grafting a silane expressed as Y—R2-Si(OR′)n, the silane S is grafted onto the probe N through the reactive group Y. This reactive group Y may be electrophilic or nucleophilic, and it reacts with a reactive group present on the probe.

The silanised probe N can then be grafted onto the sol-gel matrix by condensation of the terminal part —Si(OR′)n with residual silanols present in the preformed network of the sol-gel matrix. This grafting may also be done by incorporation of the silanised biomolecule into the deposited and dried layer (dry gel). In this case, it depends on diffusion of the biomolecule in the gel. Nevertheless, the high porosity of the layer enables relatively fast diffusion and grafting density.

The diffusion rate and the density of grafted probes can be increased by working before the gel is too dry, the solvent still trapped in the cavities facilitating distribution of the probe throughout the volume of the sol-gel matrix.

Finally, an attempt can be made to incorporate the silanised probe into the colloidal suspension in the liquid state, and this substance can then be deposited on the substrate. This technique can give an intimate mix on the molecular scale between the colloidal suspension and the probes, optimising the quality of the grafted probes and guaranteeing a uniform density of grafted probes throughout the volume of the layer.

One variant embodiment is firstly to silanise the sol-gel material during preparation of the colloidal suspension or after deposition of the layer, and then to incorporate the probe in the dry gel, the wet gel or the colloidal suspension. Grafting is then done using an organic condensation reaction.

The choice between the two variants will be different for each case and will depend on the ease of grafting of:
silane onto the probe,
silane or the silanised probe onto the sol-gel matrix,
the probe onto the silanised sol-gel matrix.

When grafting an organic sequence R1 onto the probe, the organic sequence must comprise two reactive groups Y1 and Y2. The reactive group Y2 will react with a reactive group X1 of the probe, while the reactive group Y2 will react with a reactive group X2 of the sol-gel material. Therefore the organic sequence R1 will thus be used as a coupling agent between the sol-gel matrix and the biomolecule.

The reactive group X2 will be one of the groups mentioned above in the description of the inorganic/organic composite sol-gel layers.

In the same way as for silanisation, the method of grafting R1 to the biomolecule and to the sol-gel matrix can be chosen to optimise the efficiency and the final density.

Grafting of oligonucleotides probes N onto a sol-gel layer requires several steps.

Firstly, the oligonucleotide probes N are hydroxylated in a basic solution. They are then silanised with a compound derived from a silane triethoxy to form Si—O—Si bonds between the substrate and the silane in the probe. The terminal function of the silane is chosen so that it can bond covalently to a modified oligonucleotide in 5′ for example by an amino group (for example, an aldehyde could be chosen).

Finally, the oligonucleotides are covalently grafted onto the layer of sol-gel material. In this example embodiment, 20-mer probes modified in the 5′ position by an NH2 arm are deposited in solution in a 0.3M phosphate buffer with a concentration of 10 μm.

Probes deposited on the sol-gel layers are hybridised with targets with 0.1 μM concentration of a complementary sequence carrying the fluorophore group CY3 in the 5′ position.

When the biochip has been produced, its fluorescence is observed on a GS 3000 confocal scanner. In measuring the fluorescence signal originating firstly from the hybridised probes on glass according to the state of the art, and secondly from the biochip produced according to the invention (in other words a glass substrate comprising a sol-gel thin layer), it is observed quantitatively that glass slides treated with a sol-gel layer have a gain of 1.5 to 2 compared with glass according to the state of the art.

### BIBLIOGRAPHY

[1] RAMSAY G., “DNA chips: state of the art”, Nature Biotechnol., 16, 40-44 (1998).
[2] PEASE A. C. and al., Proc. Natl. Acad. Sci., USA 91, 5022-5026 (1994).
[3] Patent WO94277191A1, issued on Aug. 12, 1994.
[4] Patent FR9902819, filed on Aug. 03, 1999.
[5] E. D. SOUTHERN, V. MASKOS, NAR 21, 2267 (1993).
[6] ZAMMATTEO N., Anal. Biochem, 253, 180-189 (1997).
[7] SCHENA M., Science, 270, 467-470 (1995).
[8] ALLEMAND J. F., Biophys. J., 73, 2064-2070 (1997).
[9] JOOS B., Natur Genet. (suppl), 21, 10-14 (1997).
[10] RASMUSSEN S. R., Anal. Biochem., 198, 138-142 (1991).
[11] SCHENA M., Proc. Natl. Acad. Sci., USA 93, 10614-10619 (1996).
[12] LAMTURE J. B., NAR 22, 2121-2125 (1994).
[13] MicamTM CEA-LETI: Réalisation de puces à ADN par electropolymérisation localisée de sondes oligonucléotidiques greffées sur un polymère conducteur (Production of DNA chips by local electropolymerisation of oligonucleotidic probes grafted onto a conducting polymer).
[14] KRAPKO K. A., FEBS Letters., 258, 118-122 (1989).
[15] YERSHOV G., Proc. Natl. Acad. Sci. (1996).
[16] EdN Timofeev, NAR 24, No. 16, 3142-3148 (1996).
[17] MIRZABEKOV A., Annal Biotec 2000 Feb. 15, 278(2):123-31.
[18] MIRZABEKOV A., Biotechniques 2000 Oct. 29(4):844-8, 850-2.
[19] U.S. Pat. No. 5,981,734, <<Methods for immobilizing nucleic acids on a gel substrate>>.
[20] BRENNAN J. D., Applied spectrocopy, 53-3 (1999).
[21] BRENNAN J. D., Chem Materials, 12, 3695-3704 (2000).
[22] KHAN I., Biochemistry, 39, 16099-16109 (2000).
[23] LAN E. H., Chem Mat, 12, 1874-1878 (2000).
[24] PREININGER C., Talenta, 52, 921-930 (2000).
[25] CHIA Sn, J. Am. Chem. Soc., 122, 6488-6489 (2000).
[26] BARRICAULT P. et al., Applied Optics, 41, No.22, 4732-4738, August 2002.

1. Biochip support comprising a substrate supporting at least one porous layer of material on a first face, the said layer being designed to fix biological molecules onto the said layer and in the volume of this layer, the said support being characterized in that the said layer is a thin optical layer of material prepared by the sol-gel method and for which the refraction index is less than the refraction index of the substrate.

2. Biochip support according to claim 1, characterized in that it also comprises at least one optical layer of material prepared by a sol-gel method supported by a second face of the substrate opposite the first face, the said thin layer supported by the second face having a refraction index lower than the refraction index of the substrate.

3. Biochip support according to claim 1, characterized in that it comprises a stack of dielectric thin layers forming a Bragg mirror inserted between the substrate and the thin layer of material prepared by the sol-gel method.

4. Biochip support according to claim 1, characterized in that the substrate is formed from a material chosen from among the group comprising glasses, polymers and semiconductors.

5. Biochip support according to claim 1, characterized in that the material prepared by the sol-gel method has a purely inorganic composition.

6. Biochip support according to claim 1, characterized in that the material prepared by the sol-gel method is composed of an inorganic fraction and an organic fraction.

7. Biochip support according to claim 6, characterized in that the inorganic fraction is larger than the organic fraction.

8. Biochip support according to claim 6, characterized in that the inorganic fraction confers its cohesion to the sol-gel material.

9. Biochip support according to claim 5, characterized in that the said material comprises at least one compound chosen from among:
an oxide MxOy, where M is chosen from among the group composed of Si, Al, Zr, Ti and Ta,
an -M-O-M′- type compound, where M and M′ are chosen from among the group composed of Si, Al, Zr, Ti and Ta.

10. Biochip support according to claim 9, characterized in that when the material prepared by the sol-gel method comprises an -M-O-M′- type compound, M is Si and M′ is Zr or Ti.

11. Biochip support according to claim 6, characterized in that the organic fraction is a polymer, the said polymer remaining free or being weakly bonded to the elements forming the inorganic fraction.

12. Biochip support according to claim 6, characterized in that the organic fraction is the result of incorporating a silane X—R2-Si(OR1)n into the inorganic fraction.

13. Biochip support according to claim 12, characterized in that:
R1 is chosen from among the group comprising —CH3, —C2H5, nPr, iPr or tBu,
R2 is an aliphatic chain with length p-CH2, preferably without an ether function —CH2-O—CH2-, where p is between 2 and 10,
X is a reactive terminal organic group chosen from among the group comprising —OH, —COOH, —CH═O, —NH2, —Cl, -epoxy, -glycidoxy, —CH═CH2, -acryl or -methalcryl.

14. Biochip support according to claim 1, characterized in that the said thin layer of material prepared by the sol-gel method has pores with size of between 5 nm and 100 nm, and a total porosity of between 1 % and 50%.

15. Process for grafting biological molecules or biomolecules onto and into the thin layer of material prepared by the sol-gel method on the first face of the biochip support according to claim 1, characterized in that it comprises the following steps:
a sol is prepared that will provide the sol-gel material,
biomolecules are incorporated into the material during its preparation,
biomolecules are grafted into the material during its preparation,
a thin layer of the said sol is deposited on the first face of the substrate,
the thin layer of sol-gel material is obtained starting from the thin layer of sol.

16. Grafting process according to claim 15, characterized in that the biomolecules incorporated into the material during its preparation are silanised biomolecules so that they can be grafted.

17. Grafting process according to claim 16, characterized in that biomolecules are incorporated into the said thin layer by diffusion when it is in the form of a dry gel.

18. Grafting process according to claim 16, characterized in that biomolecules are incorporated into the said thin layer when it is in the form of a wet gel, the biomolecules being grafted while the gel is drying.

19. Grafting process according to claim 16, characterized in that biomolecules are incorporated to the sol-gel material when it is in the form of sol, biomolecule grafting being made in the sol before deposition of the thin layer in the liquid state.

20. Grafting process according to claim 15, characterized in that the preparation step of the sol includes a functionalisation step to obtain a functionalised sol-gel material for grafting biomolecules after they have been incorporated in the thin layer.

21. Grafting process according to claim 20, characterized in that the biomolecules are incorporated into the thin layer when the thin layer is in the form of a dry gel.

22. Grafting process according to claim 20, characterized in that the biomolecules are incorporated into the thin layer when the thin layer is in the form of a wet gel.

23. Grafting process according to claim 20, characterized in that the biomolecules are incorporated in the sol-gel material when the material is in sol form, the biomolecules being grafted in the sol before deposition of the thin layer.

24. Grafting process according to claim 20, characterized in that the biomolecules are also functionalised, and are then incorporated and grafted in the sol before the sol is deposited in a thin layer.

25. Grafting process according to claim 15, characterized in that it also comprises a step for structuring the thin layer of sol-gel material to obtain a network of pads or wells over all or part of the biochip support.

26. Grafting process according to claim 25, characterized in that the said pads or wells have a characteristic dimension of between 10 and 200 micrometers, and are at a spacing of 50 to 200 micrometers.

27. Process according to claim 25, characterized in that the network of pads or wells is made using at least one of the techniques chosen from among etching, peeling, micro-machining of the layer of material prepared by the sol-gel method or by direct deposition of a structured layer of material prepared by the sol-gel method by local micro-distributions.

28. Biochip support according to claim 6, characterized in that the said material comprises at least one compound chosen from among:
an oxide MxOy, where M is chosen from among the group composed of Si, Al, Zr, Ti and Ta,
an -M-O-M′- type compound, where M and M′ are chosen from among the group composed of Si, Al, Zr, Ti and Ta.

29. Biochip support according to claim 6, characterized in that when the material prepared by the sol-gel method comprises an -M-O-M′- type compound, M is Si and M′ is Zr or Ti.
